(19) United States
(12) Patent Application Publication
(10) Pub. No.: US 2003/0064953 A1
(43) Pub. Date: Apr. 3, 2003

(54) Method for transforming diaphragm muscle cells

(76) Inventors: Feng Liu, Pittsburgh, PA (US); Leaf Huang, Wexford, PA (US)

Correspondence Address: KIRKPATRICK & LOCKHART LLP, 535 SMITHFIELD STREET, PITTSBURGH, PA, 15222, US

(21) Appl. No.: 10/191,798

(22) Filed: Jul. 9, 2002

Related U.S. Application Data
(63) Continuation of application No. 09/696,691, filed on Oct. 25, 2000.

(51) Int. Cl. A61K 48/00

(52) U.S. Cl. 514/44; 606/191

(57) ABSTRACT

An in vivo method is provided for transforming diaphragm muscle fibers. The method includes the steps of introducing into diaphragm vasculature a transforming nucleic acid, typically by intravenous delivery, and inhibiting blood flow through the diaphragm for at least about one second. This method finds particular use in the transfer of genes to the diaphragm muscle fibers. An in vivo method for transferring a dystrophin gene to diaphragm is also provided. Lastly, a non-human animal, typically a mammal is provided having diaphragm muscle fibers transformed according to the methods described herein.

### BACKGROUND

1. Technical Field

A method for transforming diaphragm muscle cells in vivo is provided. A method for introducing nucleic acid encoding a dystrophin gene into diaphragm muscle fibers also is provided. Lastly a non-human animal having transformed diaphragm muscle fibers is provided.

2. Description of the Related Art

Duchenne muscular dystrophy (DMD) and the milder allelic Becker muscular dystrophy (BMD) are X-linked genetic disorders (Emery, A. E. H. in Oxford Monographs on Medical Genetics (Oxford Medical Publications, Oxford, 1993); incorporated herein by reference). DMD is caused by the absence of dystrophin (Zubrzcha-Gaam, E. E. et al., The Duchenne muscular dystrophy gene product is localized in the sarcolemma of human muscle, Nature 333, 466-469 (1998); incorporated herein by reference). Dystrophin is a 427-kd protein encoded on the short arm of the X chromosome by the largest gene currently known (Koenig, M. et al., Complete cloning of the Duchenne muscular dystrophy (DMD) cDNA and preliminary genomic organization of the DMD gene in normal and affected individuals, Cell 50, 509-517 (1987); incorporated herein by reference). Dystrophin is a cytoplasmic protein that does not contain any transmembrane domains; it is tightly associated with a group of transmembrane proteins and actin filaments of the cytoskeleton (Matsumura, K. & Campbell, K. P., Deficiency of dystrophin-associated proteins: a common mechanism leading to muscle cell necrosis in severe childhood muscular dystrophies, Neuromuscular Disord. 3, 109-118 (1993); Ervasti, J. M., Ohlendieck, K., Kahl, S. D., Graver, M. G. & Campbell, K. P., Deficiency of a glycoprotein component of the dystrophin complex in dystrophic muscle Nature 15, 595-606 (1994); both of which are incorporated herein by reference). This dystrophin containing network links the actin cytoskeleton to the extracellular matrix (Engel, A. G., in Myology, Basic and Clinical, McGraw-Hill, N.Y., 1994; incorporated herein by reference). Because of its essential location, dystrophin has been attributed with several functions, including regulation of sarcolemmal permeability, mechanical protection against the shear forces occurring during myofiber contraction, and contribution to the regulation of calcium influx and efflux (Hoffman, E. P. & Gorospe, J. R., in Ordering the Membrane Cytoskeleton Trilayer, Academic Press, San Diego, (1992); Hoffman, E. P. & Schwartz, L., Dystrophin and disease, Mol. Aspects Med. 12, 118-119 (1991); McArdle, A., Edwards, R. H. & Jackson, M. J., How does dystrophin deficiency lead to muscle degeneration?—evidence from the mdx mouse, Neuromusc. Disord. 5, 445-456 (1995); each of which are incorporated herein by reference). DMD is characterized by progressive muscular atrophy and degeneration with concomitant loss of function and affects approximately 1 in 3,500 male newborns (Kunkel, L. M. & Hoffman, E. P., Duchenne/Becker muscular dystrophy: a short overview of the gene, the protein, and current diagnostics, Br. Med. Bull. 45, 630-643 (1989); incorporated herein by reference). The disease presents with proximal muscle weakness and may result in a delay of motor development milestones. Affected boys are wheelchair-bound at approximately 10 years of age and usually die from respiratory failure in the third decade (Emery, (1993)).

A potential cure for DMD is the delivery of the normal dystrophin cDNA to affected tissue. In attempts to develop this therapeutic strategy, direct gene transfer into muscle has been evaluated in animal models. Reporter gene and full-length or truncated dystrophin have been successfully introduced into the hindlimb muscle of mdx mice using various experimental approaches, including viral vectors (Dunckley, M. G., Wells, D. J., Walsh, F. S. & Dickson, G., Direct retroviral-mediated transfer of a dystrophin minigene into mdx mouse muscle in vivo, Hum. Mol. Genet. 2, 717-723 (1993); Ragot, T. et al., Efficient adenovirus-mediated transfer of a human minidystrophin gene to skeletal muscle of mdx mice, Nature 361, 647-650 (1993); Vincent, N. et al., Long-term correction of mouse dystrophic degeneration by adenovirus-mediated transfer of a minidystrophin gene, Nat. Genet. 5, 130-134 (1993); each of which are incorporated herein by reference) and intramuscular injection of plasmid DNA (Acsadi, G. et al., Human dystrophin expression in mdx mice after intramuscular injection of DNA constructs, Nature 352, 815-818 (1991); Danko, I. et al., Dystrophin expression improves myofiber survival in mdx muscle following intramuscular plasmid DNA injection, Hum. Mol. Genet. 2, 2055-2061 (1993); Davis, H. L. & Jasmin, B. J. Direct gene transfer into mouse diaphragm, FEBS Lett. 333, 146-150 (1993); Decrouy, A. et al., Mini-dystrophin gene transfer in mdx4cv diaphragm muscle fibers increases sarcolemmal stability, Gene Ther. 4, 401-408 (1997); each of which are incorporated herein by reference).

Although the results are encouraging, particularly with adenovirus and naked plasmid DNA, these available systems are associated with some drawbacks that have limited their application for the treatment or cure of DMD. It has been demonstrated that intramuscular injection of adenoviral vector encoded dystrophin has resulted in a transient, robust expression of dystrophin in muscles at the injection site. However, no long-term and wide-spread expression can be obtained with this vector (Blau, H. M. Muscular dystrophy, Muscling in on gene therapy, Nature 364, 673-675 (1993); Morgan, J. E., Cell and gene therapy in Duchenne muscular dystrophy, Hum. Gene Ther. 5, 165-173 (1994); both of which are incorporated herein by reference). Furthermore, the immunological and inflammatory activities of the adenoviral vector prohibit repeated administration (Lochmuller, H. et al., Transient immunosuppression by FK506 permits a sustained high-level dystrophin expression after adenovirus-mediated dystrophin minigene transfer to skeletal muscles of adult dystrophic (mdx) mice, Gene Ther. 3, 706-716 (1996); Clemens, P. R et al., In vivo muscle gene transfer of full-length dystrophin with an adenoviral vector that lacks all viral genes, Gene Ther. 3, 965-972 (1996); both of which are incorporated herein by reference). On the other hand, delivery of naked DNA to skeletal muscle has demonstrated a long-term gene expression (Wolff, J. A. et al., Direct gene transfer into mouse muscle in vivo, Science 247, 1465-1468 (1990); Wolff, J. A., Ludtke, J. J., Acsadi, G., Williams, P. & Jani A., Long-term persistence of plasmid DNA and foreign gene expression in mouse muscle, Hum. Mol. Genet. 1, 363-369 (1992); both of which are incorporated herein by reference). However, delivery of naked DNA to muscles requires local intramuscular injection that cannot achieve transduction levels sufficient for human trials. Since respiratory failure and subsequent death result in part from progressive diaphragm muscle degeneration, a potential treatment paradigm is direct gene transfer into the diaphragm. Compared with hindlimb muscles, the muscle in the diaphragm is poorly accessed by direct injection due to its location, small size and thickness. Also, direct injection can result in only localized gene expression and possible damage to the muscle fibers in the diaphragm (Davis et al. (1993)). Because of these drawbacks, systemic administration of genes to the diaphragm is likely to be required for successful delivery.

It is therefore desirable to develop an efficient method of gene transfer into the diaphragm through systemic administration of naked plasmid DNA. Previously, it has been shown that increasing the retention time of plasmid DNA in certain tissue results in enhanced gene transfer (Song, Y. K., Liu, F. and Liu, D., Enhanced gene expression in mouse lung by prolonging the retention time of intravenously injected plasmid DNA, Gene Ther. 5, 1531-1537 (1998); incorporated herein by reference). In that article, retention time of plasmid DNA only in lung tissue was modulated by the preinjection of liposomes into the test animals. Nevertheless, an approach is desirable that successfully allows the uptake and expression of naked plasmid DNA in the muscle cells (fibers) of the diaphragm following intravenous injection of the plasmid DNA without the need for the administration of additional compounds or compositions. It is also desirable to deliver a full-length and functional dystrophin gene to the diaphragm.

### SUMMARY

A method for transforming diaphragm muscle fibers in vivo, is provided. This biotechnological method includes the steps of introducing a transforming nucleic acid into diaphragm vasculature and inhibiting blood flow through the diaphragm for an amount of time sufficient to permit transformation of the muscle fibers by the transforming nucleic acid. The transforming nucleic acid is preferably naked in that no vector, delivery vehicle or other transformation- or transduction-facilitating compound or composition, such as liposome or viral capsid, is needed to transfer the nucleic acid into the diaphragm muscle fibers. The transforming nucleic acid typically is DNA. The transforming nucleic acid may be administered into the diaphragm systemically, typically intravenously, or into arteries that supply the diaphragm. The transforming nucleic acid typically is administered in a pharmaceutically acceptable aqueous composition that includes the nucleic acid and, typically, pharmaceutically acceptable buffer(s) and salt(s). A method for transferring a dystrophin gene into diaphragm muscle fibers also is provided.

Also provided is a non-human animal, typically a mammal and more typically one of a mouse, a rat, a rabbit, a cat, a dog, a pig, a sheep, a cow or a horse, having diaphragm muscle fibers transformed with a nucleic acid according the above-described method. Such animals find use as models for certain disease states and in the production of non-native proteins, typically secreted proteins.

### BRIEF DESCRIPTIONS OF THE DRAWINGS

FIGS. 1A and 1B are graphs showing the tissue distribution and time-dependence of luciferase gene expression in the diaphragm after tail vein injection of 80 μg of pNGVL-Luc DNA in 100 μl saline. The venous drainage of the diaphragm was blocked by clamping the inferior vena cava immediately next to the diaphragm for 8 seconds immediately following the injection. In FIG. 1A, the liver, diaphragm, spleen, kidney, lung, and heart of injected mice were collected 1 day after the injection and assayed for luciferase activity. In FIG. 1B, for time-dependent gene expression, the mice were sacrificed at the indicated time after injection. The data are expressed in RLU (relative light units) per mg of total protein extracted from the tissue as mean±S.D. (n=3).

FIGS. 2A and 2B are graphs that show the effect of clamp time and DNA dose on the level of luciferase gene expression in the diaphragm. In FIG. 2A, blood flow through the diaphragms of mice was blocked from 1 to 16 sec immediately following tail vein injection of 80 μg plasmid DNA (pNGVL-Luc) in 100 μl of saline. The level of luciferase gene expression in the diaphragm of each mouse was determined 1 day after injection. In FIG. 2B, various amounts of plasmid DNA in 100 μl saline were tail vein injected into each mouse. To occlude blood flow through the diaphragm the inferior vena cava next to the diaphragm was clamped for 8 sec immediately following the injection. The level of gene expression was measured 6 days after injection. Data represent mean±S.D. (n=3).

FIGS. 3A-3D show immunohistochemical staining for dystrophin in the diaphragm muscle from an untreated mdx mouse (FIG. 3A), a C57 normal mouse (FIG. 3B), a mdx mouse 3 days after gene transfer (FIG. 3C), and an mdx mouse 7 days after gene transfer (FIG. 3D). Magnification: 400×.

FIGS. 4A-4C show hematoxylin and eosin (H & E) staining and FIGS. 4D-4F show Evans Blue fluorescence of 10 μm cryosections from the diaphragm muscles. H & E staining is shown for a C57 mouse (FIG. 4A), an mdx mouse (FIG. 4B), and an mdx mouse 7 days after dystrophin gene transfer (FIG. 4C). Evans Blue fluorescence is shown in a C57 mouse (FIG. 4D), an mdx mouse (FIG. 4E), and an mdx mouse 7 days after dystrophin gene transfer (FIG. 4F). H & E staining and Evans Blue fluorescence were visualized by phase contrast and fluorescence microscopy, respectively. Magnification: 200×.

### DETAILED DESCRIPTION

A method is provided for the transformation of diaphragm muscle fibers. The described method is superior to prior art methods because there is no need for the administration of any compound or composition to the diaphragm muscle fibers other than a transforming nucleic acid in water or saline and no need for ancillary nucleic acid sequences in the nucleic acid molecule other than those necessary for the transforming activity of the nucleic acid. The method includes the steps of administering a transforming nucleic acid into diaphragm vasculature (blood vessels) and inhibiting the flow of blood through the diaphragm for a short period of time. Muscle fibers of the diaphragm are efficiently transformed by this method and express transformed protein for long periods of time.

By the term “transforming nucleic acid,” it is meant any nucleic acid, or an analog thereof, that can elicit an effect in a cell. “Nucleic acids” include, for example and without limitation, DNA, including antisense DNA; RNA, including mRNA, antisense RNA and ribozymes; and oligonucleotides, including peptide nucleic acids, phosphorothioates and methyl phosphonates. Typically, the transforming nucleic acid is deoxyribonucleic acid (DNA). The transforming nucleic acid does not necessarily have to integrate into the genome of the transformed muscle fibers, and does not have to remain active in its transforming effect in the cell for any given length of time. So long as the transforming nucleic acid elicits a desired effect in a transformed muscle fiber for any length of time, it is considered to be a “transforming nucleic acid.” Typically, the “transforming nucleic acid” includes a gene for expression in the diaphragm muscle fibers. The gene is expressed in the transformed muscle fiber to produce a desired RNA and/or protein product. Typically, it is desirable to realize long-term transformation of the cell, i.e. longer than about one week, as opposed to transient transformation. Depending on the desired transformation effect, integration of the “transforming nucleic acid” into the muscle cell genome may be desirable.

Additional non-transforming nucleic acid sequences may be present in the transforming nucleic acid, such as, without limitation, sequences of the vector used to propagate the transforming nucleic acid, such as bacterial, plasmid, viral, phage and yeast artificial chromosome (YAC) sequences. Prior to use in the described methods, the administered transforming nucleic acid may be isolated and purified from the vector nucleic acid used to propagate the transforming sequence. This may be done by standard molecular biological methods.

When the nucleic acid includes a gene for expression in the diaphragm muscle fibers, the gene includes suitable expression control sequences (i.e., without limitation, transcription control sequences such as, without limitation, promoters, enhancers and terminators), operatively linked to form a transcription unit. Promoters can be, for example and without limitation, constitutive or semi-constitutive (e.g., RSV and CMV promoters) or tissue-specific promoters (e.g., a muscle creatinine kinase (MCK) promoter). Use of muscle-specific promoters, such as the MCK promoter, as opposed to semi- or fully-constitutive promoters such as the CMV and RSV promoters, may be preferred when the nucleic acid is administered systemically, to target expression of the protein in diaphragm muscle fibers to prevent expression in other tissues. By using a muscle-specific promoter, systemic dissemination of the nucleic acid and the potentially harmful or undesirable transient and/or long-term expression of the nucleic acid in organs other than the diaphragm, may be prevented.

As used herein, any derivative, analog, or homolog of dystrophin, which retains dystrophin functionality capable of correcting or alleviating the symptoms of DMD or BMD is considered to be “dystrophin.” Therefore, without limitation, native dystrophin, alleles thereof, engineered versions thereof, nonhuman analogs or homologs thereof or mutants thereof containing insertions, deletions, replacements and modifications, including post translational modifications (collectively “functional derivatives” of dystrophin) are considered to be “dystrophin,” so long as dystrophin activity is retained.

As shown in the embodiments described below, the method of the present invention has been used to transfer a marker gene (firefly luciferase) and a therapeutic gene (dystrophin) into diaphragm muscle cells. However, there are a variety of applications for the method of the present invention. First, the method may be used to correct genetic defects or to overcome the effects of diseases that affect the diaphragm. As with the case of DMD, other diseases affect the diaphragm. Myopathics, such as myopathies with myosin loss, can affect thediaphragm. If the disease can be corrected by transforming the diaphragm with a gene or genes, such as a dominant allele or alleles, it can be corrected by the method of the present invention, provided the gene for the dominant allele has been characterized to a sufficient degree. If the expression of a gene has been inhibited in the diaphragm, or is not at a level sufficient to achieve normal diaphragm function, the gene may be supplemented by the method of the present invention. Lastly, abnormal over-expression of a gene may be corrected by transforming muscle fibers with nucleic acids encoding known inhibiting factors, antisense sequences or protein-binding proteins or peptides, such as immunoglobulin fragments, such as single chain Fᵥ fragments.

Diseases that are not of the diaphragm, nevertheless may be treated by the method of the present invention. The diaphragm muscle fibers may be transformed with a gene that expresses or indirectly causes the expression of a secreted factor (for example, secreted proteins such as, without limitation, Factor VIII) in a patient deficient in that secreted factor, so that a defect caused by the deficiency of that factor may be corrected. Secreted proteins may be produced that elicit effects elsewhere in the patient, such as hormone secretagogues. So long as the production of the secreted protein by the transformed diaphragm does not substantially affect respiratory function, this treatment method may be desirable.

Transformation of the diaphragm of non-human animals also is desirable. The production of secreted factors in non-human animals has substantial commercial prospects. Such secreted factors may include recombinant antibodies or peptide drugs. The ability to use these animals as sources of these compounds is desirable. As above, so long as the transformation of the diaphragm of these animals does not substantially affect respiratory function in these animals, the method of the present invention may be used to this end. Typically, the animal is mammalian and may include, without limitation mice, rats, rabbits, cats, dogs, pigs, sheep, cows, horses and monkeys.

Dominant defects also can be introduced into non-human animals by the method of the present invention by the transformation of the diaphragm by a dominant defective allele of a gene. Animals carrying these defects are useful in drug screening, for instance. This technique is especially desirable when the defect is lethal or requires that animals carrying the defect be maintained in a special manner. In such a case, it may be desirable to transform an unaffected animal by the method of the present invention rather than maintaining a defective line by transgenic or other somatic methods. The method described herein is quite easy, and robust animals may be used. Through the use of other recombinant technologies, such as antisense techniques, recessive defects also may be introduced. Defects in muscle cell metabolism, or defects in cellular metabolism that are common to all cells, including muscle cells, may be preferred.

As described above, to achieve the desired transformation of the diaphragm, the transforming nucleic acid must be introduced into the diaphragm vasculature. This may be achieved by a number of routes. Introduction of the transforming nucleic acid into the diaphragm vasculature is most easily achieved by administering the transforming nucleic acid systemically, and typically intravenously. Other routes for the administration of the transforming nucleic acid may be desirable, as discussed in further detail below.

To achieve transformation of the diaphragm, blood flow through the diaphragm is inhibited when the transforming nucleic acid is present in the diaphragm vasculature. Any method know to be capable of inhibiting blood flow through the diaphragm may be used, and inhibition of blood flow may be initiated at any time prior to or after the introduction of the transforming nucleic acid into the diaphragm vasculature, so long as the blood flow is inhibited when the nucleic acid is within the diaphragm vasculature. Typical methods inhibit the blood flow through the diaphragm at least partially. One method for inhibiting blood flow through the diaphragm is by occluding blood vessels that supply or drain the diaphragm. Occluding blood flow through the diaphragm may be accomplished by clamping or otherwise blocking one or more vessels, and typically all vessels, supplying or draining the diaphragm, including without limitation, phrenic veins, the inferior vena cava downstream from the phrenic veins (between the phrenic veins and the heart so that blood flow from the phrenic veins, and therefore the diaphragm, is inhibited), phrenic arteries and the aorta. The veins and/or arteries may be clamped by standard surgical methods, or even by hand. The veins and/or arteries also may be partially or fully occluded using a catheter suitably configured and administered to occlude the one or more veins or arteries that either drain or supply the diaphragm. The catheter may be a balloon catheter or other similar instrument. Use of a balloon catheter to inhibit blood flow may be preferable in humans and in non-human animals of sufficient size, since the catheter surgery is minimally invasive.

In one embodiment of the method described herein, a sufficient quantity of transforming nucleic acid is administered intravenously and, immediately thereafter, blood flow from the diaphragm is inhibited for about eight seconds by clamping the inferior vena cava downstream from the phrenic veins.

Despite the ease of systemic administration of the transforming nucleic acid, systemic administration of the transforming nucleic acid may be undesirable for a number of reasons. For instance, systemic administration in larger animals is likely to require large quantities of nucleic acid. Further, systemic administration of a particular nucleic acid may cause undesirable side effects and toxicities. Therefore, it may be preferable to administer the transforming nucleic acid locally into the diaphragm. This may be achieved by first inhibiting the blood flow through the diaphragm by occluding the arteries feeding the diaphragm and/or the veins draining the diaphragm and subsequently injecting the transforming nucleic acid into an artery that supplies the diaphragm, and preferably into arteries that exclusively supply the diaphragm. For instance, in one embodiment, the arteries feeding the diaphragm are occluded and the transforming nucleic acid is subsequently injected into arteries that supply the diaphragm at a point between the occlusion and the diaphragm. In an alternative embodiment, the veins draining the diaphragm are occluded prior to injection of the transforming nucleic acid into arteries that supply the diaphragm. In a further embodiment, both the arteries feeding the diaphragm and the veins draining the diaphragm are occluded prior to injection of the transforming nucleic acid into the arteries that supply the diaphragm at a point between the arterial occlusion and the diaphragm. In each of these alternate embodiments, after a desired time sufficient to permit transformation of the diaphragm muscle fibers, all occlusions are removed.

The blood flow through the diaphragm may be inhibited for any medically or veterinarily reasonable length of time, so long as the length of time is sufficient to permit the transformation of the muscle fibers by the nucleic acid. Nevertheless, once the transforming nucleic acid is introduced into the diaphragm vasculature, the blood flow need not be inhibited for more than a few seconds. Once the transforming nucleic acid is introduced into the diaphragm vasculature, the blood flow through the diaphragm typically is inhibited at a minimum from about 1 to 4 seconds, inclusive, and at a maximum from about 8 seconds to about 16 seconds, preferably from about 4 to about 8 seconds. The optimal time period may vary from species-to-species, and can be determined empirically.

The nucleic acid is administered as a pharmaceutical or veterinary composition that includes other inactive ingredients that facilitate the given delivery method. These other ingredients, which are useful in, for example and without limitation, preserving or delivering the nucleic acids, are referred to collectively herein as “excipients.” Specific examples of excipients include, without limitation, buffers, salts, proteins or peptides, fats or lipids, polymeric materials, dyes and sugars. Solutions containing the nucleic acid may be stored or packaged in sealed vessels or syringes and may form part of a kit that includes other items, such as instructional pamphlets, to facilitate distribution of and end-use of the nucleic acid.

### EXAMPLES

Materials

Plasmid pNGVL-Luc containing the cDNA of firefly luciferase driven by the cytomegalovirus (CMV) promoter was custom prepared by Bayou Biolabs (Harahan, La., USA). The plasmid DNA encoding the full-length murine dystrophin cDNA under control of the SRα promoter (pSRα-dys) was used (Clemens, P. R. et al., Recombinant truncated dystrophin minigenes: construction, expression, and adenoviral delivery, Hum. Gene Ther. 6, 1477-1485 (1995); incorporated herein by reference). Plasmid DNA was prepared according to standard methods. The Luciferase assay kit was from Promega (Madison, Wis., USA). CD-1 male mice (15 g) were from Charles River Laboratories (Wilmington, Mass., USA). Six-week-old mdx mice (C57BL/10ScSndmd-mdx) were bred from stock mice purchased from Jackson Laboratories. 2,2,2-tribromoethanol was purchased from Aldrich Chem. Co. (Milwaukee, Wis., USA). C57BL/10 mice (C57 mice) were obtained from Charles River Laboratories.

Methods

Gene Delivery Methods

Mice were anesthetized with an intraperitoneal injection of 8 mg of 2,2,2-tribromoethanol. The diaphragm was exposed through a ventral midline incision. Mice were intravenously (tail vein) injected, with luciferase or full-length murine dystrophin plasmid DNA in 100 μl saline (0.9% NaCl) for one to two seconds. Immediately after injection, blood flow through the diaphragm was occluded for 8 seconds by placing a clip (a hemostat modified with silicon tubing over its teeth to prevent damage to the clamped blood vessel) on the inferior vena cava below the diaphragm (the junction of the hepatic vein and caudal vena cava).

Protein Assays

The mice were sacrificed at different time points and their diaphragms were removed. Lysis buffer was added to each diaphragm (0.5 ml of 0.1% Triton X-100, 2 mM EDTA, and 0.1 M Tris-HCl pH 7.8) and the diaphragm was homogenized by using a Tissue Tearor and centrifuged at 14,000 rpm for 2 min. A 10 μl aliquot of the supernatant was analyzed for luciferase activity. Luminescence was measured for 10 sec for each assay, and the luciferase activity for the each assay was presented as relative light units per mg of total extracted protein in the tissue (RLU/mg protein).

Immunofluorescence Analysis

The diaphragm was dissected from treated mdx mice at the defined time point. Frozen sections (10 μm) were made using a Jung Frigocut (Leica, Germany). The sections were preincubated for 1 h at room temperature with 10% horse serum in PBS (pH 7.4) and then incubated overnight with affinity purified sheep anti-dystrophin antibody d10 (a gift from E. P. Hoffman) (Koenig, M. and Kunkel, L. M., Detailed analysis of the repeated domain of dystrophin reveals 4 potential hinge regions that may confer flexibility, J. Biol. Chem. 265, 4560-4566 (1990); Hoffman, E. P., Morgan, J. E., Watkins, S. C. and Partridge, T. A., Somatic reversion/suppression of the mouse mdx phenotype in vivo, J. Neurol. Sci. 99, 9-25 (1990); both of which are incorporated herein by reference). After four rinses in 10% horse serum/PBS, the sections were incubated with biotinylated donkey anti-sheep antibody (Jackson Immunoresearch Laboratories, 1:250 dilution) for 1 h followed by 30-min incubation with Cy3-conjugated streptavidin (Jackson Immunoresearch Laboratories, 1:5000 dilution). As controls, the diaphragm sections from C57BL/10 and untreated mdx mice were similarly processed.

For H & E staining, 10 μm sections were stained for 5 min in hematoxylin and 30 sec in eosin, dehydrated with ethanol and xylene, and mounted with Vectashield mounting medium (Vector Laboratories, Inc., Burlingame).

In vivo Membrane Permeability Assay

Evans blue dye injection and microscopic analysis were performed as described previously (Straub, V., Rafael, J. A., Campbell, J. S, and Campbell, K. P., Animal models for muscular dystrophy show different patterns of sarcolemmal disruption, J. Cell. Biol. 139, 375-385 (1997); incorporated herein by reference). Evans Blue was dissolved in phosphate-buffer saline (10 mg/ml) and sterilized by passage through membrane filters with 0.2 μm pore size. Mice were intravenously injected with 0.5 mg dye per 10 g body weight. The mice were sacrificed at 4 h after dye injection. Diaphragm muscle sections (10 μm) were incubated in ice-cold acetone at −20° C. for 10 min, washed 3×10 min with PBS, and mounted with Vectashield mounting medium. All sections were examined and photographed with a Nikon fluorescence microscope (Nikon Corp., Japan).

Results

Tissue Distribution and Time-Course of Gene Expression

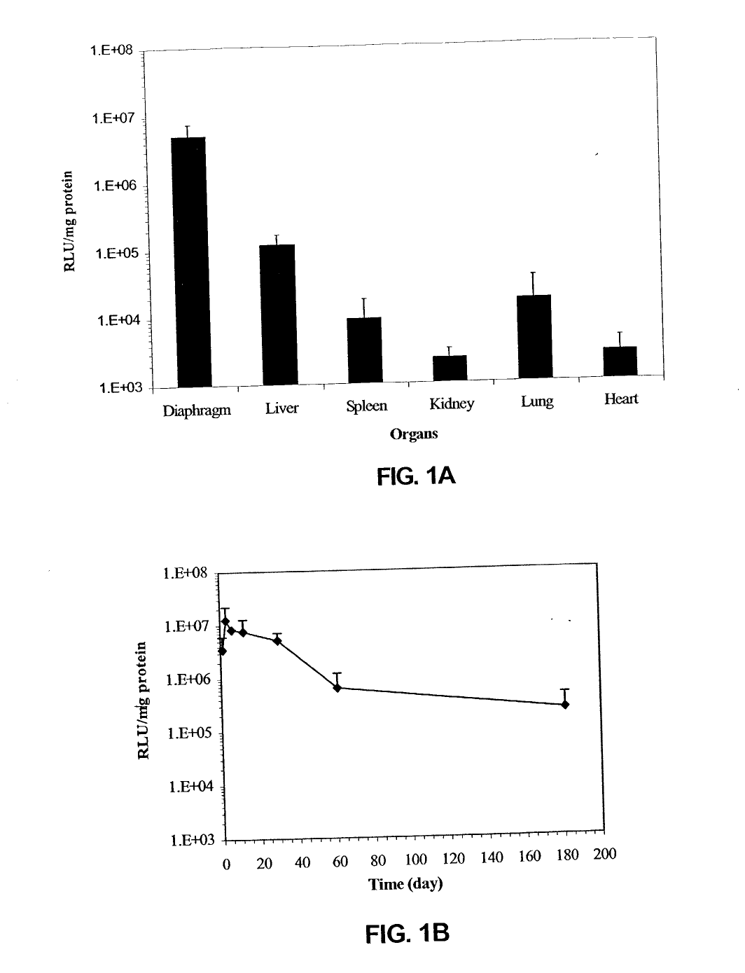

The blood outflow through the diaphragm was blocked by clamping the inferior vena cava for 8 seconds immediately following intravenous injection of 80 μg of luciferase plasmid DNA in CD1 mice. The level of gene expression in different organs including liver, lung, spleen, heart, kidney, and diaphragm was first examined. As shown in FIG. 1A, the diaphragm expressed the highest level of luciferase activity among all the tested organs, approximately 5×10⁶ RLU (Relative Light Unit) per mg of total extracted protein 24 hours after administration of luciferase plasmid DNA. Gene expression in other organs was much less; approximately 50 fold lower in the liver and 300-1500 fold lower in lung, spleen, kidney, and heart. Time-dependent gene expression shown in FIG. 1B indicates the level of luciferase activity in the diaphragm reached a peak level of 1.3×10⁷RLU/diaphragm on day 6, followed by a slow decline of gene expression. The luciferase activity dropped only approximately 1.5 fold from day 3 to day 30, and decreased another 8 fold from day 30 to day 61. The luciferase activity at 2 months after injection was 6.5×10⁵ RLU per diaphragm of mouse injected with 80 μg luciferase plasmid DNA, and this level of luciferase was persistently expressed for at least six months. The level of gene expression in other organs including the liver, lung, heart, spleen, and kidney was undetectable at day six after administration.

Effect of Clamp Time and DNA-Dose on Gene Expression

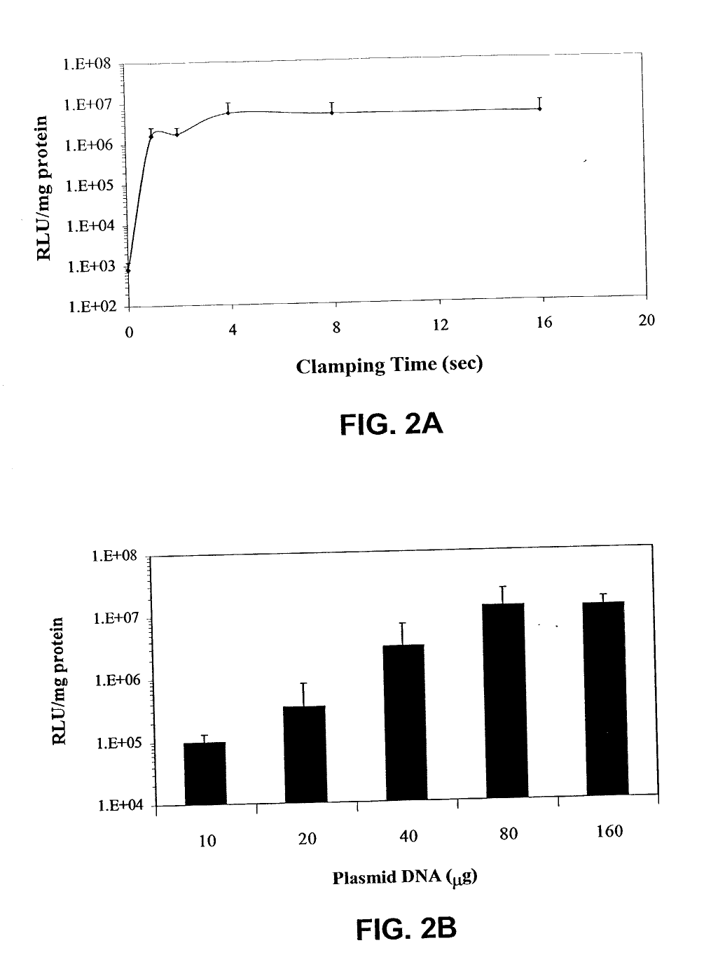

The effect of clamping time on gene expression was investigated. The blood outflow through the diaphragm was occluded by clamping the inferior vena cava from 1 to 16 seconds and the level of luciferase activity was detected 24 hours after injection of the DNA. As shown in FIG. 2A, there was little or no gene expression detected in the diaphragm without clamping. However, a significant level of luciferase protein was obtained 24 hours after injection when the vena cava clamping time was as short as 4 sec. Further prolongation of the time of occlusion did not improve gene expression. The effect of occlusion time on gene transfer could be related to effects of the occlusion of blood flow on the physiologic condition of the diaphragm. This in turn could facilitate gene uptake by the diaphragm muscle. This possibility was investigated by clamping blood flow via the vena cava for 8 seconds immediately before, rather than immediately after, the DNA injection. In this case, no gene expression in the diaphragm was observed (data not shown). This indicates that the enhanced gene expression in the diaphragm was not due to any physiological changes in the diaphragm resulting from the occlusion of blood flow. The dose effect of DNA on the level of gene expression also was measured. The level of luciferase protein activity in the diaphragm was detected one day after injection of different amounts of DNA in 100 μl saline followed by clamping the vena cava for 8 seconds. FIG. 2B shows that gene expression correlated with the dose of plasmid DNA. The luciferase activity in the diaphragm increased with increasing the amount of plasmid DNA injected and reached a saturation level at 80 jig plasmid DNA per mouse.

Full-Length Dystrophin Expression in mdx Mice

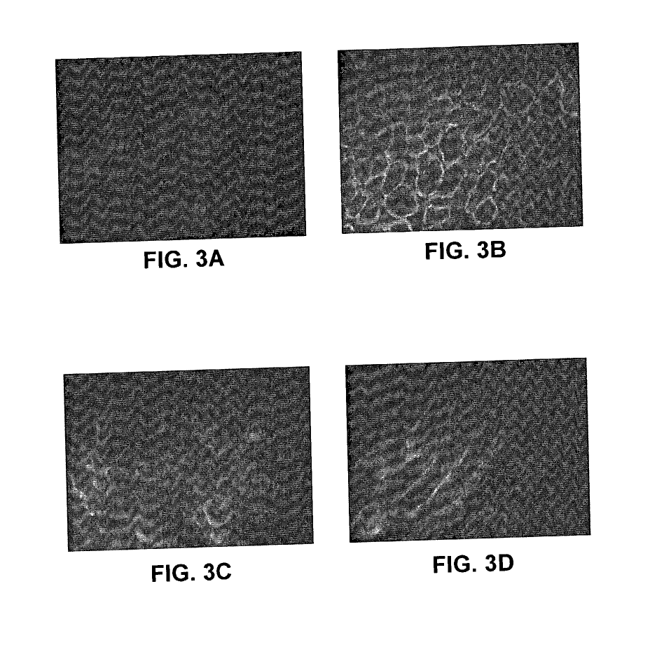

Murine dystrophin expression was determined in mdx mice following intravenous injection of 400 μg (approximately 1.5 fold molar equivalent of 80 μg of luciferase plasmid) of plasmid containing the full-length murine dystrophin cDNA driven by the SRα promoter. The representative result of immunostaining for dystrophin in the normal wild-type mice (C57), mdx mice, or mdx mice transduced with the dystrophin transgene are shown in FIGS. 3A-3D. Only background anti-dystrophin immunofluorescence was seen in the diaphragm of an untreated mdx mouse (FIG. 3A), confirming the specificity of the antibody (negative control). Dystrophin immunostaining in a C57 mouse showed membrane staining in all muscle fibers (FIG. 3B). In contrast, membrane staining of a number of fibers was observed in the diaphragm sections taken from a mdx mouse 3 days after the transfection (FIG. 3C). In addition, dystrophin-positive fibers were also detected in a longitudinal section taken from the diaphragm sample of an injected mdx mouse 7 days after injection (FIG. 3D). It was estimated that approximately 15-20% of muscle fibers were dystrophin-positive. The distribution of fibers expressing recombinant dystrophin was heterogeneous. The reasons for this observation are not fully understood.

Functional Rescue of Dystrophin

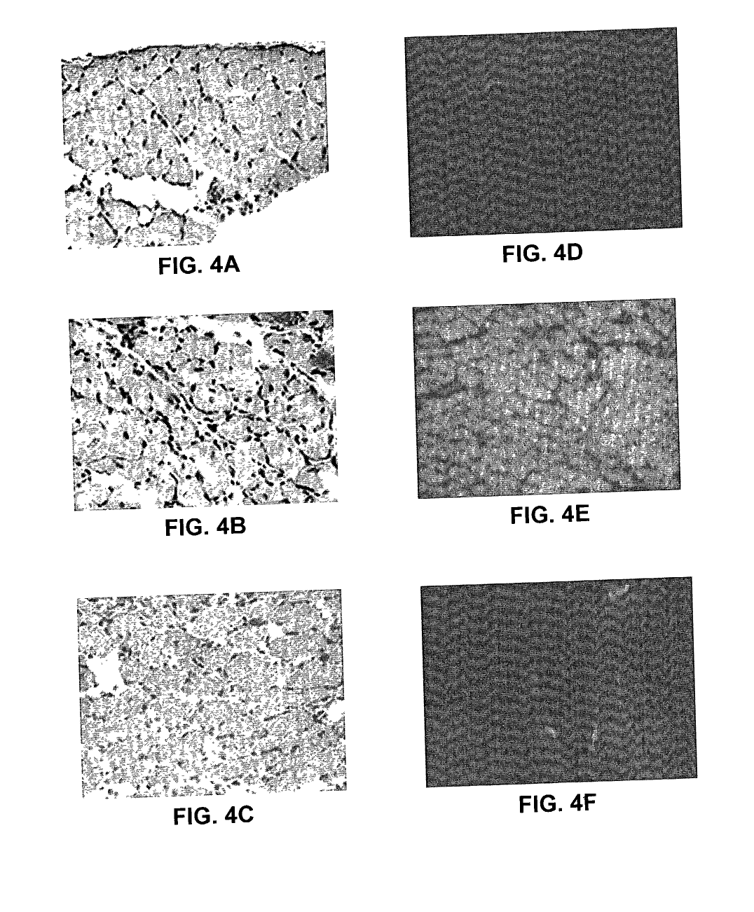

One way to assess the functional rescue achieved by dystrophin delivery is to determine whether the restoration of dystrophin in the diaphragm muscle will prevent the histopathological process of sarcolemmal damage and muscle degeneration. Central nucleation in muscle fibers is a sign of continued cycles of degeneration and regeneration due to muscle cell damage. As shown in FIG. 4, the diaphragm muscle fibers stained with H&E showed peripheral nuclei for normal mouse (FIG. 4A), and high levels of centralized nuclei in an mdx mouse (FIG. 4B). However, the number of centralized nuclei in vector-transduced diaphragm muscle was significantly less 7 days after dystrophin gene transfer when compared to age-matched, untreated mdx control (FIG. 4C). This finding suggests that the expression and assembly of dystrophin in the cell membrane of the diaphragm fibers have prevented the process of muscle degeneration.

Furthermore, intracellular accumulation of an impermeable dye can be used to monitor sarcolemmal membrane integrity in mdx mice. To assess the level of sarcolemmal damage in diaphragm fibers of treated and untreated mdx mice, mdx mice were injected with Evans Blue dye. Dye incorporation into muscle fibers was clearly visible in the diaphragm of an untreated mdx mouse (FIG. 4E). In contrast, significantly less dye incorporation was observed in diaphragm fibers from an mdx mouse 7 days after dystrophin gene transfer (FIG. 4F). Control C57 mouse diaphragm muscle demonstrated no dye incorporation (FIG. 4D). Together, these results provide strong evidence that dystrophin has been restored in the cell membrane in the mdx diaphragm muscle fibers and is functioning to stabilize sarcolemmal integrity using the new gene transfer technology.

Respiratory failure is often the ultimate cause of death in DMD patients. One reason for respiratory failure is progressive muscle degeneration in the diaphragm that results from the absence of dystrophin. Therefore, gene transfer into the diaphragm has attracted increasing attention as a component of the treatment approach for this disorder. Previously diaphragm gene transfer was performed by local intramuscular injection (Davis et al. (1993); Petrof, B. J. et al., Efficiency and functional consequences of adenovirus-mediated in vivo gene transfer to normal and dystrophic (mdx) mouse diaphragm, Am. J. Respir. Cell Mol. Biol. 13, 508-517 (1995), incorporated herein by reference). Clinical application of this technique is limited by the fact that gene expression was found only over a small area with a radius of 1-2 mm from the injection site (Karpati, G., Pari, G. and Molnar, M. J., Molecular therapy for genetic muscle diseases—status, Clin. Genet. 55, 1-8 (1999); incorporated herein by reference). Therefore, direct intramuscular delivery would require numerous injections even for a single diaphragm muscle, which could cause damage to the diaphragm.

The results reported here represent the first demonstration of gene transfer into the diaphragm muscle via intravenous injection of naked plasmid DNA without using any carrier system (viral or non-viral vectors), or physical force (direct injection, electroporation, and/or hydrodynamic pressure). Satisfactory gene transfer is achieved by simple occlusion of blood flow through the diaphragm following intravenous administration of luciferase or fill-length dystrophin plasmid DNA. The method is safe and causes no detectable damage to myofibers. Up to 1 ng of luciferase protein per mg of extracted protein was obtained from the diaphragm of a mouse following a single injection of plasmid DNA in 100 μl saline. A significant level of gene expression was detected even 6 months following vector administration.

Dystrophin expression in normal skeletal muscle is concentrated at those regions subjected to the highest levels of longitudinally and radially transmitted mechanical stress. The stresses are transmitted to the membrane during muscle contraction (Petrof, B. J., The molecular basis of activity-induced muscle injury in Duchenne muscular dystrophy, Mol. Cell. Bioch. 179, 111-123 (1998); incorporated herein by reference). It has been demonstrated that dystrophin plays an important role in mechanical protection against the shear forces during myofiber contraction (McArdle et al. (1995)). Therefore, one of the central issues concerning gene therapy for DMD is whether gene transfer can accomplish dystrophin expression over a sufficient extent of the muscle fiber membrane to provide a functional benefit. Immunostaining of diaphragm sections using an anti-dystrophin antibody demonstrated the sarcolemmal localization of the dystrophin gene expression in transduced mdx mice: dystrophin transgene expression was observed not only in cross-sections of the muscle fibers but also in longitudinal sections (FIG. 3). It should be noted that while only 15-20% of muscle fibers in the diaphragm were detected to be dystrophin-positive, the number of transfected cells might exceed that number, given the limited sensitivity of the immunostaining technique. Previous studies using germ-line dystrophin cDNA delivery in transgenic mice have suggested that when the dystrophin level is restored to approximately 20% of the normal, the specific force generated by the diaphragm muscle is not significantly different from that of control normal mice (Phelps, S. F. et al., Expression of full-length and truncated dystrophin mini-genes in transgenic mdx mice, Hum. Mol. Genet. 4, 1251-1258 (1995); incorporated herein by reference). Thus, the level of gene expression achieved by the gene delivery strategy described herein should provide a therapeutic effect. Indeed, the histology study described herein demonstrated that dystrophin gene delivery to the diaphragm led to an improvement in histopathology (FIG. 4). The diaphragm in mdx mice undergoes degeneration with centralized nuclei. This pattern was significantly improved following dystrophin gene transfer. In addition, as assessed by intracellular uptake of Evans Blue dye, the loss of membrane integrity in the diaphragm was significantly ameliorated by dystrophin gene transfer.

The mechanism of plasmid DNA uptake achieved by the systemic delivery approach described herein is not clear. Increase in the number, size, and permeability of the miccrovascular pores has been proposed as the mechanism in pressure-based muscle gene transfer (Budker, V., Zhang, G., Danko, I., Williams, P. and Wolff, J., The efficient expression of intravascularly delivered DNA in rat muscle, Gene Ther. 5, 272-276 (1998); incorporated herein by reference). In that reference intra-arterial delivery of plasmid DNA to muscle could be greatly enhanced when the DNA is injected rapidly, in a large volume, and with all blood vessels leading into and out of the hindlimb occluded. In Budker et al., the authors injected 9.5 ml of DNA solution into one hindlimb of the rat within 10 sec in order to generate the essential pressure. Based on this observation, one could argue that gene transfer to the diaphragm follows a similar pressure-based mechanism. Diaphragmatic intravascular pressure may be elevated as a function of occluding blood outflow, without altering cardiac output, which could have a net effect of enlarging pores for DNA entry. In conflict with this interpretation, however, rapid intravenous injection was performed with 10% of body weight of DNA solution to generate pressure that is high enough to achieve gene transfer to the internal organs (Liu, F., Song, Y. K. and Liu, D., Hydrodynamics-based transfection in animals by systemic administration of plasmid DNA., Gene Ther. 6, 1258-1266 (1999); Zhang, G. Budker, V. and Wolff, J. A., High levels of foreign gene expression in hepatocytes after tail vein injections of naked plasmid DNA, Hum. Gene Ther. 10, 1735-1737 (1999); both of which are incorporated herein by reference), and observed no enhancement in gene expression in the diaphragm (data not shown).

Another postulated mechanism of plasmid DNA uptake is by a receptor-mediated or a nonspecific binding/uptake process. Previous work in this area was largely done by Wolff's group in isolated hepatocytes (Budker, V. et al., Hypothesis: naked plasmid DNA is taken up by cells in vivo by a receptor-mediated process, J. Gene Med. 2, 76-88 (2000); incorporated herein by reference). They suggest that naked DNA may be taken up by a receptor-mediated process, although the receptor has not been identified. Furthermore, the putative receptor is said to bind DNA with low affinity. Little information is available for muscle, particularly with systemically delivered DNA. Due to the rapid blood flow and low affinity of the putative receptor, DNA may not remain in close proximity with a given cell surface receptor long enough to result in endocytosis. Since the affinity of DNA to its receptor is weak, a “successful” binding will require a relatively long contact time of DNA with the receptor, a situation precluded by rapid blood flow. Hypothetically, if flow is stopped, even briefly, binding and endocytosis might be increased. Consequently, DNA might have a greater opportunity to be taken up and expressed by diaphragm muscle cells. A similar situation has been shown in the interaction of a mobile ligand and its receptor (Rosenfeld, R., Vajda, S. and Delisi, C., Flexible docking and design, Ann. Rev. Biophys. Biomol. Struct. 24, 677-700 (1995); Jackson, R. M., and Sternberg, M. J. E., A continuum model for protein-protein interaction: Application to the docking problem, J. Mol. Biol. 250, 258-278 (1995); Weng, Z. and Delisi, C., Toward a predictive understanding of molecular recognition, Immunol. Rev. 163, 251-266 (1998); each of which are incorporated herein by reference). Nevertheless, further studies are needed to verify this hypothesis.

In conclusion, for the first time it has been demonstrated that the diaphragm can be efficiently transfected by temporarily blocking venous drainage from the diaphragm following systemic administration of naked plasmid DNA. In addition to nucleic acids, the methods described herein may be utilized to deliver proteins and other pharmaceutical compositions to the diaphragm. This technique is simple, highly reproducible and does not induce toxicity to the diaphragm. In a clinical setting, occlusion of blood outflow from the diaphragm might be performed by a balloon catheter (Stephan, D. J. et al., A new cationic liposome DNA complex enhances the efficiency of arterial gene transfer in vivo, Hum. Gene Ther. 7, 1803-1812 (1996); incorporated herein by reference) instead of a surgical procedure. This simple procedure may represent an important advance toward gene therapy of DMD and BMD.

The above invention has been described with reference to the preferred embodiment. Obvious modifications and alterations will occur to others upon reading and understanding the preceding description and the claims. It is intended that the invention be construed as including all such modifications and alterations.

We claim:

1. A method for transforming diaphragm muscle fibers in vivo, comprising the steps of:
a. introducing a transforming nucleic acid into diaphragm vasculature; and
b. inhibiting blood flow through the diaphragm for a period of time sufficient to permit transformation of the muscle fibers by the transforming nucleic acid.

2. The method of claim 1, wherein the transforming nucleic acid is introduced to the diaphragm vasculature by the systemic administration of the transforming nucleic acid.

3. The method of claim 2, wherein the transforming nucleic acid is administered systemically by intravenous injection.

4. The method of claim 1, wherein the inhibiting step comprises the step of occluding a vein that drains the diaphragm.

5. The method of claim 4, wherein the vein is the inferior vena cava, which is occluded downstream from a phrenic vein.

6. The method of claim 4, wherein the vein is occluded prior to the step of administering the transforming nucleic acid into the diaphragm vasculature.

7. The method of claim 6, wherein the transforming nucleic acid is introduced into the diaphragm vasculature by introducing the transforming nucleic acid into an artery that supplies the diaphragm.

8. The method of claim 1, wherein the inhibiting step comprises the step of occluding an artery that supplies the diaphragm.

9. The method of claim 8, wherein the transforming nucleic acid is introduced into the diaphragm vasculature after the blood flow through the diaphragm is inhibited.

10. The method of claim 9, wherein the transforming nucleic acid is introduced into the diaphragm vasculature by injecting the nucleic acid into an artery supplying the diaphragm between the arterial occlusion and the diaphragm.

11. The method of claim 8, wherein the inhibiting step further comprises the step of occluding a vein that drains the diaphragm.

12. The method of claim 11, wherein the transforming nucleic acid is introduced into the diaphragm vasculature after the blood flow through the diaphragm is inhibited.

13. The method of claim 12, wherein the transforming nucleic acid is introduced into the diaphragm vasculature by injecting the nucleic acid into an artery supplying the diaphragm between the arterial occlusion and the diaphragm.

14. The method of claim 1, wherein the inhibiting step is performed using a catheter suitably configured to occlude a blood vessel that supplies or drains the diaphragm.

15. The method of claim 14, wherein the catheter is a balloon catheter.

16. The method of claim 1, wherein the transforming nucleic acid includes a gene for expression in the diaphragm muscle fibers.

17. The method of claim 16, wherein the gene encodes dystrophin.

18. The method of claim 16, wherein the gene encodes an antisense RNA.

19. The method of claim 1, wherein, after the nucleic acid is introduced into the diaphragm vasculature, the blood flow through the diaphragm is inhibited for a period of time ranging from about 1 to about 16 seconds.

20. The method of claim 19, wherein, after the nucleic acid is introduced into the diaphragm vasculature, the blood flow through the diaphragm is inhibited for a period of time ranging from about 4 to about 8 seconds.

21. A method for expressing a dystrophin gene in a diaphragm, comprising the steps of:
a. introducing into diaphragm vasculature a nucleic acid containing a dystrophin gene for expression in diaphragm muscle cells; and
b. inhibiting blood flow through the diaphragm for a period of time sufficient to permit transformation of the muscle fibers by the nucleic acid.

22. The method of claim 21, wherein the blood flow is inhibited by occluding a vein that drains the diaphragm and/or an artery that supplies the diaphragm.

23. The method of claim 22, wherein, after the nucleic acid is introduced into the diaphragm vasculature, the blood flow through the diaphragm is inhibited for a period of time ranging from about 1 to about 16 seconds.

24. The method of claim 23, wherein, after the nucleic acid is introduced into the diaphragm vasculature, the blood flow through the diaphragm is inhibited from about 4 to about 8 seconds.

25. A non-human animal in which a diaphragm muscle fiber of the animal is transformed according to the method of claim 1.

26. The non-human animal of claim 25, wherein the animal is a mammal.

27. The non-human animal of claim 26, wherein the mammal is selected from the group consisting of mouse, rat, rabbit, cat, dog, pig, sheep, cow, horse and monkey.

28. The non-human animal of claim 26, wherein the mammal is a mouse.
